(19) United States
(12) Patent Application Publication
(10) Pub. No.: US 2021/0212695 A1
(43) Pub. Date: Jul. 15, 2021

(54) BLOOD VESSEL SAMPLE KIT

(71) Applicant: Dr. Shobghana Sugirthamuthu, Dharmapuri (IN)

(72) Inventors: SHOBHANA SUGIRTHAMUTHU, Dharmapuri (US); PARIKUMAR PERIASAMY, Dharmapuri (IN)

(21) Appl. No.: 17/250,362

(22) PCT Filed: Jul. 13, 2018
(86) PCT No.: PCT/IB2018/055182

§ 371 Date: Jan. 11, 2021

(30) Foreign Application Priority Data
Oct. 27, 2017 (IN) 201741038179

(51) Int. Cl. A61B 17/122; A61B 17/02; A61B 17/32

(52) U.S. Cl. CPC A61B 17/122; A61B 2017/320064; A61B 17/32; A61B 17/0231

(57) ABSTRACT

This invention pertains to the field of medical diagnostics which is used to take a sample of blood vessel, called BLOOD VESSEL SAMPLE, for investigation purposes. The blood vessel sample kit composed of flexible clips attached with the stand, having tooth on the lower surface on either side of the clips. The blood vessel sample kit also having a triple scissors with a spring handle and three sets of upper and lower blades, purposed to cut off a small portion of the blood vessel, to make the vascular endothelium exposed, easy for culturing. The blood vessel sample is taken from the VISIBLE VASCULAR WEB of the SUPRA TENON VASCULAR WEB. The purpose of the blood vessel sample taking is to study the VASCULAR ENDOTHELIAL RESPONSE TO DIABETES—VERD, which is a part of conjunctival vasculopathy.

### BACKGROUND OF THE INVENTION

Everyone is familiar with blood sample taken by the health professionals for investigations by subjecting blood and its components to various tests. Blood has got two components called cellular component and serum component. Serum is the clear fluid obtained once the cells are separated from the blood. Both the blood cells and the clear fluid are used for various investigations to assess the health of the individual from whom blood sample is taken. But our concept in this invention is based on obtaining a blood vessel sample for investigation purposes. The blood vessel sample taken from a human being will give us more valuable information on the health of that individual. The inner lining of the blood vessel called ENDOTHELIUM is a single cell layer lining the entire blood vessel net work throughout the body. Where ever there is blood in the body, that blood is carried by the blood vessel.

The major human diseases like micro vascular abnormalities of diabetes and hypertension, Heart attack, stroke and all other end organ diseases are due to the problems associated with blood and the blood vessel. The problems in the blood are addressed so far and the problems associated with the blood vessel are not addressed so much.

This invention pertains to the field of medical diagnosis, particularly dealing with the problems associated with the blood vessel. Just like blood sample taken from the body is being subjected to different tests to assess the health of the individual, the BLOOD VESSEL SAMPLE could also be taken from the body to assess the health of the individual. We investigate and treat the person based on the abnormalities found in the blood and make it normal. But we don't pay much attention to the blood vessel, the pipe line that carries blood. Any leakage of blood or any blockage of the blood vessel leads to serious health hazards. More than 50% of human diseases are due to the abnormalities in the blood and blood vessel.

This blood vessel sample is proposed to be obtained from the conjunctiva of the eye, which is an elastic tissue present on the visible part of the sclera and also present on the inner aspect of the lids. It is folded at the fornix. Hence it is distinctly called as bulbar conjunctiva, forniceal conjunctiva and palpebral conjunctiva. The bulbar conjunctiva and the forniceal conjunctiva have high elasticity allowing the rotational movement of the eye ball. The whole conjunctival tissue is enriched with a rich vascularity containing largely the venules, minute arterioles, met arterioles and capillaries. The arterioles are branching from the ophthalmic artery and the venules are the tributaries of ophthalmic veins. The micro circulation happens in most part of the conjunctiva. The venules with the inner lining of endothelium are the focus of this observation. Most of the venules are empty and invisible most of the times under normal circumstances. In the event of an injury, the venules which were invisible become visible and the lumen of the blood vessels become patent and within seconds of the injury gets filled with blood and remains so causing what is commonly known as RED EYE, until the inflammation subsides. In some situations the inflammation comes under control in a few minutes and in other situations it takes several weeks or months to come under control. Once the inflammation comes under control, again the empty and invisible blood vessels revert to the original condition and remain so. Here the empty and invisible blood vessels having the endothelial cell lining the inside of them naturally are touching the opposite endothelial cells but there seems to be no permanent adhesions since during injury there seems to be no difficulty in the empty blood vessels being filled with blood. This we call—

Invisible Vascular Web.

The invisible vascular web becomes conspicuous only when provoked.

The blood vessels which normally appear within the conjunctiva constitute the

Visible Vascular Web The visible vascular web could be easily divided into

SUPRA TENON VASCULAR WEB and

INFRA TENON VASCULAR WEB, based on the anatomical location of the vascular web.

The vascular webs appear as an overlapped image when observed under the slit lamp. The supra tenon vascular web appears as superimposed over the infra tenon vascular web. This is the normal conjunctiva in physiological conditions. The repeatedly inflamed conjunctiva is distorted and looses this appearance. Conjunctivochalasis and other common inflammatory conditions enormously alter this appearance. The supra tenon vascular web is the one which offers the opportunity to provide a

BLOOD VESEEL SAMPLE, which could be taken with utmost ease.

The uniqueness of each individual displays the ability of the creator to create ‘n’ number of designs in His creation. Commonly we use the finger impressions and iris pattern to identify human beings but there is a lot more information unique for each individual. In our opinion, each human cell is unique and each human cell responds to the external atmosphere in an unique way. The categorization we make based on ethnicity and geographical differences are very vague and unacceptable by scientific terms. We conduct studies with a reasonable ‘p’ value and justify ourselves of the results and implement them on the society. The common man has no choice but to accept the treatment given to him based on the outcome and inference of the results of our studies. Because of the flaws in our studies, the conclusion of some of our studies actually give very negligible amount of information on the subject concerned but the outcome is implemented on the masses. We are not justified in doing so.

We need to understand the fact that from human cell to the galaxies, the creator has a say in each unit of the creation. In other words, the uniqueness is existing from human cells to the galaxies. Each cell is different and each star is different.

So, recommending medical treatment based on our studies may be a misleading venture. In this light, if the treatment studies on diabetes are viewed, we will come to understand that something is going wrong somewhere. Here we attempt to provide a clue to probably getting the right perspective.

### FIELD OF INVENTION

This invention pertains to the field of medical diagnostics, where a blood vessel sample is taken from the body, from the conjunctiva with the help of the device explained in this invention, the Blood vessel sample Clip and The triple cutter having three prongs to prepare the blood vessel sample ready for cell culture. The central cutter is meant to cut the blood vessel on the top portion that is at the 12 o'clock position of the lumen of the blood vessel. The cutter on either side is meant to cut the conjunctiva attached to the blood vessel at its proximal aspect. A single pressing of the triple cutter spring will cut the blood vessel and the conjunctiva on either side making an open exposed endothelial surface and a portion of the conjunctiva on either side of the blood vessel. This blood vessel cut portion along with the portions of conjunctiva on either side is placed on the culture plate or culture medium and sent to the laboratory.

### PRIOR ART

The prior art in this specialty is that the blood sample only is taken from the body for investigation purposes to assess the health status of the individual. The blood vessel taken from the body for investigation purpose is the new concept and hence there is no prior art. The sample or the body tissue taken for biopsy is entirely different issue and that is not the scope of this invention.

### PURPOSE OF THE INVENTION

The micro vascular complications of diabetes are the real challenge. The endothelial cell response to the blood and its contents decide the life of the micro vascular stability. Our proposal is based on the fact that the endothelial cell response to the sugar levels in the blood varies from person to person and it is unique for each individual.

VERD—Vascular Endothelial Response to Diabetes

Thus we suggest that a sample of a blood vessel, about 1 mm length be taken from the body, separate the endothelial cells, culture the cells and expose the cells to different levels of sugars and find out the response for each concentration of sugars and make a standard protocol. The VASCULAR ENDOTHELIAL RESPONSE to DIABETES—VERD scale of grading is suggested. Keeping the standard scale, each person's VERD reading could be compared and treated. This means that, we are treating the patient not only on the sugar levels in the blood but also considering the VERD of each person which is highly variable.

This concept emerges from the fact that a person with 20 years history of diabetes shows no micro vascular change but a person with history of 2 years of diabetes shows enormous micro vascular changes. There are so many incomparable factors exist to explain the difference in the micro vascular changes. But one thing neglected or ignored so far in the DIFFERENCE in VERD of each individual. In my understanding, nowhere in the world, a blood vessel is taken from the patient to treat a diabetic patient.

The micro vascular changes happening in the conjunctival vasculature due to diabetes, hypertension and other chronic diseases are termed here as DIABETIC CONJUNCTIVAL VASCULOPATHY.

The blood vessel sample could be normally obtained from the VISIBLE VASCULAR WEB of the SUPRA TENON VASCULAR WEB as the INVISIBLE VASCULAR WEB appears only when the eye is provoked or inflamed. Taking a blood vessel sample from the INFRA TENON VASCULAR WEB will be more cumbersome and involves more tissue damage.

Hence the blood vessel sample provides with the vascular endothelium for the purposes of studying the ability and health of the endothelium, which determines the longevity of life.

In addition to this, the NITRIC OXIDE produced by the vascular endothelium is a very vital chemical that determines the health of human beings. The estimation of Nitric oxide production measured by the presence of Nitrates will give valuable information on the health. This also is possible with this concept of Blood Vessel Sample taking.

Ophthalmologist's Role

Getting a 1 mm sample of a blood vessel is a simple job for an ophthalmologist. Thanks to the elasticity of the conjunctiva and the blood vessels of the conjunctiva. It shrinks well after taking a 1 mm sample and healing of the epithelium of the conjunctiva happens in 24 hours.

The mushrooming stem cell laboratories around the globe make this concept an easily achievable goal.

Once a person is diagnosed of Diabetes, he can be subjected to the VERD test and based on the comparison with the standards, the patient could be treated. This treatment will be sensible in the sense that it takes in to account
1. The blood sugar levels and
2. The VASCULAR ENDOTHELIAL RESPONSE to DIABETES

Practicality
It may appear to be practically impossible to achieve this for the masses and it may appear to be possible for the ones who are gifted with access to secondary medical care. This need not be true. If planned and executed well, this could become like one as we perform culture sensitivity test. This test need not be cumbersome or too complicated.
This approach will surely PERSONALIZE the diabetes management. With this concept, we avoid unnecessary medications for years thereby avoiding the side effects of the drugs and save the economically under privileged people's money. On the other hand, we make the diabetes management more focussed. In addition to personalizing the Diabetic care, we can evaluate the ability of the vascular endothelium to produce Nitric Oxide which will personalise the management of Cardiovascular and Neurovascular disorders there by Heart attack and Stroke could be also managed in an efficient way.

Science of the Endotelium
There are 2 kinds of endothelial cells in the body.
1. Vascular Endothelium.
2. Corneal Endothelium.
Embryological differences exist but these 2 endothelial cell lines hold human life and vision respectively.
Vascular Endothelial cells are arranged as a single cell layer all over the body and if stretched and spread, an adult's endothelial cell line will cover 6 tennis courts.
Corneal Endothelial cells are also arranged as a single cell line.
These 2 cell lines are only holding human Life and Light
In other words, The Life and Vision hang from these 2 single cell lines.
The Vascular Endothelial cells could be classified into 2 categories.
1. Intra organ VE cells.
2. Extra organ VE cells.
   The IRMA, Intra Retinal Micro vascular Abnormalities, also happen in the Conjunctival Vascular Endothelial cells.
   This concept leads to a lot of other questions, but the answer may lie in the way we see this concept
   The frequency of taking blood vessel sample and the length of the blood vessel sample may be determined with experience and proper understanding of the science behind this concept.

Standard Operating Procedure [SOP]

For Blood Vessel Sample to Treat Diabetes and Also Cardiovascular and Cerebrovascular Diseases.
A. The conjunctiva is anaesthetised with topical anaesthetic eye drops of Proparacaine Hydrochloride ophthalmic solution USP 0.5% w/v., 2 drops every 2 minutes for 10 minutes.
B. The patient's head is placed under the operating microscope with all sterile precautions.
C. The eye drape is fixed to the respective eye and an opening is made on the transparent surface of the drape corresponding to the palpebral aperture.
D. A wire speculum is applied to the eye to keep the lids apart.
E. A Lim's forceps is used to pinch a medium sized conjunctival blood vessel present on the bulbar conjunctiva and lifted up gently away from the globe.
F. The conjunctival scissors is used to trim that part of the conjunctiva along with the blood vessel, approximately 3 mm in length.
G. The conjunctiva along with the blood vessel sample is placed in a tray with normal saline.
H. The portion of the conjunctiva from where the blood vessel sample is taken retracts back to the original position and there may be a slight oozing of blood at the site of pinching.
I. Alternatively, the blood vessel sample could be taken at the OPD by using a head band type magnifying glass loupe.
J. Then the blood vessel sample is subjected to the BLOOD VESSEL SAMPLE KIT.
K. This kit has got a clip and a triple cutter device. Clip is meant to hold the conjunctiva along with the blood vessel in position so as to keep the tissue taut making it convenient for the surgeon to prepare the blood vessel sample ready for culturing the endothelial cells.
L. The clip comprises of two toothed arms which act like the toothed speculum. The clips are used to hold the edges of the conjunctiva so as to have the blood vessel at the centre and the conjunctiva is positioned in such a way that it is held taut in the desired direction of the long axis of the blood vessel corresponding with the arms of the clip.
M. The central arm of the triple cutter is positioned in the centre of the blood vessel and the central part of the scissor cuts open the blood vessel, having a thin rim of conjunctival flap on either side.
N. Once the triple cutter is punched and the blood vessel sample is ready to be placed on the culture plate.
O. Then the culture plate is sent to the laboratory.
P. The laboratory performs the following investigations in different timings and the final results are obtained.

Investigations Proposed
A. Endothelial cell count/unit area.
B. Endothelial cell morphology.
C. Endothelial cell response to varying sugar concentrations at varying timings.
D. The presence of Nitrates in the culture plate, which is an indicator of the Nitric Oxide production capacity of the vascular Endothelium.
E. Vascular endothelial growth factor analysis.
F. Any other investigation relevant to the endothelial cell health and the response to different life chemicals.

BLOOD VESSEL SAMPLE KIT

The blood vessel sample kit is composed of
1. Clip
2. Triple cutter
3. A plastic wire speculum
4. Two plastic Lims forceps with metal tip
5. A plastic Vannas scissors with metal tip
6. Ear buds 4 pieces
7. Cotton—sufficient quantity
8. A pair if universal size gloves
9. The vial containing the transport medium
10. A glass Petri dish
11. A saline ampoule
12. Endothelial stain

A screw may be used to retain the arms in position. Then the triple cutter is used to cut through the top portion of the blood vessel at 12 0' clock position so that the endothelial surface of the blood vessel is open upwards with its edges having a rim of conjunctiva on either side. Then the blood vessel sample is placed over the culture medium and the process of culturing the vascular endothelial cells is done in the laboratory. The investigations are performed at later dates depending on the need and other medical and biological compulsions.

The first two instruments are new ones and the remaining items from 3-12 are commonly available in the Ophthalmic market.

### DRAWINGS

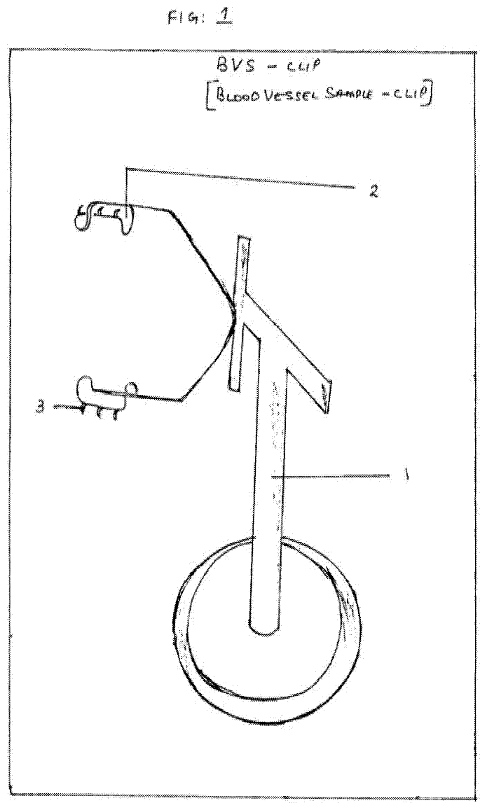

FIG. 1: BVS—CLIP Blood Vessel Sample Clip
1. Stand
2. Flexible clip
3. Tooth

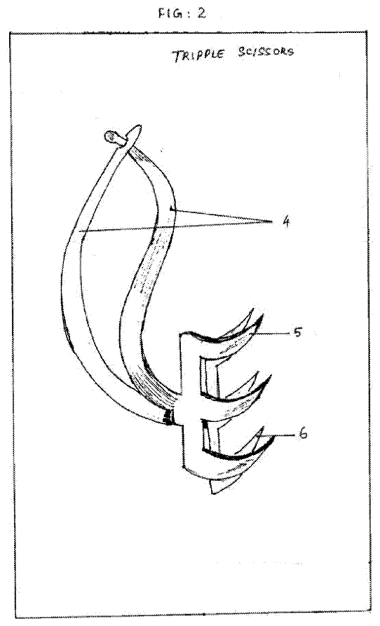

FIG. 2: TRIPLE SCISSORS
4. Spring handle
5. Upper Blades
6. Lower Blades

### DETAILED DESCRIPTION

1. The Blood vessel sample kit contains a stand 1 with a base and a supportive stand. This stand is provided with a wire speculum 2 and the clip has tooth 3 at the lower surface of the flexible clip 2 purposed to hold the conjunctiva with the blood vessel in position. This stand is kept under the microscope to provide utmost convenience to the surgeon by keeping the conjunctiva blood vessel tissue taut. The edges of the conjunctiva are trimmed in such a way that the tip of the blood vessel is facing the surgeon. Then the triple cutter is taken and the three sets of blades 5 & 6 are brought to the trimmed edge of the conjunctiva with the blood vessel at its centre. The clips are used to hold the edges of the conjunctiva so as to have the blood vessel at the centre and the conjunctiva is positioned in such a way that it is held taut in the desired direction of the long axis of the blood vessel corresponding with the arms of the clip. Once the triple cutter is pressed, the blood vessel sample is ready for culturing.

The spring handle 4 of the triple scissors is pressed to cut the blood vessel along with the remnants of conjunctiva on either side. This blood vessel sample is placed on the transport or culture medium to be sent to the laboratory for further investigations. The blood vessel sample is taken from the visible vascular web of the supra tenon vascular web, because the invisible vascular web appears only when provoked or inflamed and the infra tenon vascular web needs more intervention.

The procedure is an outpatient procedure for an ophthalmologist which will take hardly 5 minutes. Alternatively, the procedure could be performed at the operation room under conventional sterile methods. In both the cases, the procedure is going to be the same.

It will be appreciated by those skilled in the art having the benefit of this disclosure that this invention provides a blood vessel sample kit. It should be understood that he drawings and the detailed description herein are to be regarded in an illustrative rather than a restrictive manner, and are not intended to limit the invention to the particular forms and examples disclosed. On the contrary the invention includes ant further modifications, changes, rearrangements, substitutions, alternatives, design choices and embodiments apparent to those of ordinary skill in the art without departing from the spirit and scope of this invention as defined by the following claims. Thus it is intended that the following claims be interpreted to embrace all such further modifications, changes, rearrangements, substitutions, alternatives, design choices and embodiments.

We claim:

1. A blood vessel sample kit, for the purpose of obtaining blood vessel sample from human body, especially, the conjunctiva, having a clip with tooth attached with the stand and a triple scissors having a spring handle, a set of three blades, upper and lower blades.

2. According to claim 1, the blood vessel sample kit having clips with the lower surface provided with tooth so as to keep tissue in position.

3. According to claims 1 & 2, the clips having tooth with flexibility and can be stretched, the tooth purposed to hold the human tissue in taut position, the conjunctiva.

4. According to claim 1, the blood vessel sample kit having a triple scissors, having a spring handle, with three sets of blades with upper and lower sets, the spring handle once compressed will close the blades to make the cut on the tissue, and when relaxed will open the blades.

5. According to claim 1, the blood vessel sample taken from the conjunctival tissue, from the visible vascular web of the supra tenon vascular web.

6. According to claims 1, 2, 3, 4 & 5, the standard operating procedure is claimed as follows:

7. According to claim 1, the blood vessel sample kit also consists of the following items, a plastic wire speculum, Two plastic Lims forceps with metal tip, a plastic Vannas scissors with metal tip, ear buds 4 pieces, cotton—sufficient quantity, a pair if universal size gloves, the vial containing the transport medium, a glass Petri dish, a saline ampoule and the Endothelial stain

8. According to claim 1, 2, 3, 4, 5, 6&7, the STANDARD OPERATING PROCEDURE [SOP] is claimed as follows:
FOR BLOOD VESSEL SAMPLE TO TREAT DIABETES AND ALSO CARDIOVASCULAR AND CEREBROVASCULAR DISEASES.
A. The conjunctiva is anaesthetised with topical anaesthetic eye drops of Proparacaine Hydrochloride ophthalmic solution USP 0.5% w/v., 2 drops every 2 minutes for 10 minutes.
B. The patient's head is placed under the operating microscope with all sterile precautions.
C. The eye drape is fixed to the respective eye and an opening is made on the transparent surface of the drape corresponding to the palpebral aperture.
D. A wire speculum is applied to the eye to keep the lids apart.
E. A Lim's forceps is used to pinch a medium sized conjunctival blood vessel present on the bulbar conjunctiva and lifted up gently away from the globe.
F. The conjunctival scissors is used to trim that part of the conjunctiva along with the blood vessel, approximately 3 mm in length.
G. The conjunctiva along with the blood vessel sample is placed in a tray with normal saline.
H. The portion of the conjunctiva from where the blood vessel sample is taken retracts back to the original position and there may be a slight oozing of blood at the site of pinching.
I. Alternatively, the blood vessel sample could be taken at the OPD by using a head band type magnifying glass loupe.
J. Then the blood vessel sample is subjected to the BLOOD VESSEL SAMPLE KIT.
K. This kit has got a clip and a triple cutter device. Clip is meant to hold the conjunctiva along with the blood vessel in position so as to keep the tissue taut making it convenient for the surgeon to prepare the blood vessel sample ready for culturing the endothelial cells.
L. The clip comprises of two toothed arms which act like the toothed speculum. The clips are used to hold the edges of the conjunctiva so as to have the blood vessel at the centre and the conjunctiva is positioned in such a way that it is held taut in the desired direction of the long axis of the blood vessel corresponding with the arms of the clip.
M. The central arm of the triple cutter is positioned in the centre of the blood vessel and the central part of the scissor cuts open the blood vessel, having a thin rim of conjunctival flap on either side.
N. Once the triple cutter is punched and the blood vessel sample is ready to be placed on the culture plate.
O. Then the culture plate is sent to the laboratory.
P. The laboratory performs the following investigations in different timings and the final results are obtained.
Q. Alternatively, the whole procedure could be performed as an outpatient procedure in an Ophthalmology clinic or office.

9. According to claims 1, 2, 3, 4, 5 & 6, the blood vessel sample is subjected to the following investigations
INVESTIGATIONS PROPOSED
A. Endothelial cell count/unit area.
B. Endothelial cell morphology.
C. Endothelial cell response to varying sugar concentrations at varying timings.
D. The presence of Nitrites and Nitrates in the culture plate, which is an indicator of the Nitric Oxide production capacity of the vascular Endothelium.
E. Vascular endothelial growth factor analysis.
F. Any other investigation relevant to the Endothelial cell health and the response to different life chemicals.

10. According to claims 1, 2, 3, 4, 5, 6 & 7, we claim the following terminologies used to obtain the blood vessel sample,
DIABETIC CONJUNCTIVAL VASCULOPATHY,
BLOOD VESEEL SAMPLE,
BLOOD VESSEL SAMPLE KIT,
VASCULAR ENDOTHELIAL RESPONSE to DIABETES—VERD,
INVISIBLE VASCULAR WEB
VISIBLE VASCULAR WEB
SUPRA TENON VASCULAR WEB
INFRA TENON VASCULAR WEB
